(19) United States
(12) Patent Application Publication
(10) Pub. No.: US 2012/0041305 A1
(43) Pub. Date: Feb. 16, 2012

(54) LIGHT-EMITTING DYE FOR INTRAOPERATIVE IMAGING OR SENTINEL LYMPH NODE BIOPSY

(75) Inventors: Charles B. Grissom, Salt Lake City, UT (US); James M. McGreevy, Holladay, UT (US); Robert Hans Ingemar Andtbacka, Salt Lake City, UT (US)

(73) Assignee: THE UNIVERSITY OF UTAH RESEARCH FOUNDATION, Salt Lake City, UT (US)

(21) Appl. No.: 13/265,456

(22) PCT Filed: Apr. 21, 2010
(86) PCT No.: PCT/US10/31882

§ 371 Date: Oct. 20, 2011

(30) Foreign Application Priority Data
Apr. 21, 2009 (US) 61171414

(51) Int. Cl. A61B 6/00

(52) U.S. Cl. 600/431

(57) ABSTRACT

The present invention relates generally to the field of fluorescent dyes as a system useful for surgery imaging. More particularly, the present invention relates to systems, methods and kits for exciting fluorescent, phosphorescent or luminescent molecules with light from a light source and detecting the relative fluorescent, phosphorescent, or luminescent light intensity emitted from the fluorescent, phosphorescent, or luminescent molecule. Such systems may be applied as mapping agents for various surgical techniques, such as for cancer surgeries and biopsies.

### CROSS-REFERENCE TO RELATED APPLICATION

The present application claims priority to U.S. provisional patent application Ser. No. 61/171,414 filed on 21 Apr. 2009, incorporated herein by reference.

### BACKGROUND OF THE INVENTION

The present invention relates generally to the field of fluorescent dyes as a system useful for surgery imaging. More particularly, the present invention relates to systems, methods and kits for exciting fluorescent, phosphorescent or luminescent molecules with light from a light source and detecting the relative fluorescent, phosphorescent, or luminescent light intensity emitted from the fluorescent, phosphorescent, or luminescent molecule. Such systems may be applied as mapping agents for various surgical techniques, such as for cancer surgeries and biopsies.

The publications and other materials used herein to illuminate the background of the invention or provide additional details respecting the practice, are incorporated by reference.

Significance of Tumor Micro-Margin Visualization

The surgical debulking of a tumor is the most common primary treatment for many types of cancer. Most commonly, surgeons use radiographically-placed localizer wires or manual palpation to define the border between tumor tissue and healthy tissue during an operation. Even with immediate cryohistological analysis of the tumor mass by a pathologist, repeat operations for additional tissue removal are required in 25-40% of tissue-conserving breast cancer lumpectomies.

Intraoperative SLN Biopsy

Sentinel lymph node (SLN) biopsy is currently the standard of care in melanoma treatment and it is quickly becoming the standard of care in therapeutic breast cancer operations. This well established procedure is based on two recent, large, nationally enrolled studies, the National Surgical Adjuvant Breast Project B-32 (NSABP) and the American College of Surgeons Trial Z-001. Both trials evaluated the SLN concept in the axillary nodal drainage in women with operable breast cancer. The accuracy of the SLN procedure in predicting the status of the axillary nodes was measured in control group subjects who received a standard axillary dissection after the SLN biopsy. Results showed that, with experience, the surgeon located the SLN in 95-98% of patients and that the SLN accurately predicts the status of the axillary nodes in 90-95% of the cases where the SLN has been identified. Combined, the studies enrolled over 10,000 patients, and while the follow-up data is not yet complete, the procedure itself, SLN biopsy, is being adopted by breast surgeons worldwide. With 186,000 new cases of breast cancer each year, the number of surgeries to remove primary tumors and determine the status of associated lymph nodes is significant.

SLN biopsy in breast cancer patients uses a combination of two technologies that were developed to treat patients with melanoma. Two agents are used to find the SLN. Lymphazurin® is a blue dye that rapidly diffuses into the lymphatic trunks after injection around the breast tumor or in the periareolar space. After making a skin incision, the surgeon is able to see blue dye in the lymphatic trunk and follow the blue color to the SLN, and thereby find a blue lymph node that is deemed to be the sentinel node. The blue Lymphazurin® dye can diffuse throughout the operative wound making dissection and SLN identification difficult. This is especially true if the lymphatic vessels are cut. A radioactive marker is often used in combination with the blue dye to aid in identification of the SLN. A solution of Technetium-99m-labeled sulfur colloid (^{99m}Tc) with an average particle size of 100 nm is prepared by a radiopharmacist. The sulfur colloid ^{99m}Tc is injected around the tumor 2 to 4 hours before the operation. A hand-held gamma detector is used to find the radioactive nodes in the surgical field. The radioactive nodes are not always blue, and the blue nodes are not always radioactive. Furthermore, a single axilla will often contain several SLNs; the average is 2.4 radioactive and/or blue nodes per patient. This may be caused by the marker passing through the SLN, or could be due to the fact that different lymphatic trunks may drain to different lymph nodes.

Significance of Lymphatic Mapping in Melanoma Surgery

The management of regional lymph nodes in patients with clinically localized primary melanomas has been controversial. An elective lymph node dissection at the time of removal of the primary melanoma has been favored by many The proponents of elective lymph node dissection has based their opinion on the hypothesis that melanoma spreads in an orderly fashion from the primary site to regional lymph nodes and then systemically.

Thus early removal of lymph node tumor deposits may prevent subsequent systemic dissemination. See, for example, Balch et al. (“A multifactorial analysis of melanoma: III. Prognostic factors in melanoma patients with lymph node metastases (stage II).” Annals Surgery (1981), 193(3):377-88); Callery et al. (“Factors prognostic for survival in patients with malignant melanoma spread to the regional lymph nodes.” Annals Surgery (1982), 196(1):69-75); Cohen, et al. (“Prognostic factors in patients undergoing lymphadenectomy for malignant melanoma.” Annals Surgery (1977), 186(5):635-42); Gupta, T. (“Results of treatment of 269 patients with primary cutaneous melanoma: a five-year prospective study.” Annals Surgery (1977), 186(2):201-9); Morton, et al. (“Improved long-term survival after lymphadenectomy of melanoma metastatic to regional nodes. Analysis of prognostic factors in 1134 patients from the John Wayne Cancer Clinic.” Annals Surgery (1991), 214(4):491-9; discussion 9-501); Reintgen, et al. (“Efficacy of elective lymph node dissection in patients with intermediate thickness primary melanoma.” Annals Surgery 1983, 198(3):379-85); and Roses, et al. (“Prognosis of patients with pathologic stage II cutaneous malignant melanoma.” Annals Surgery (1985), 201(1): 103-7).

Four prospective randomized trials of elective lymphadenectomy have tested this hypothesis. See, for example, Balch, et al. (“Long-term results of a multi-institutional randomized trial comparing prognostic factors and surgical results for intermediate thickness melanomas (1.0 to 4.0 mm) Intergroup Melanoma Surgical Trial.” Annals Surgical Oncology (2000), 7(2):87-97); Cascinelli, et al. (“Immediate or delayed dissection of regional nodes in patients with melanoma of the trunk: a randomised trial. WHO Melanoma Programme.” Lancet (1998), 351(9105):793-6); Sim, et al. (“Lymphadenectomy in the management of stage I malignant melanoma: a prospective randomized study.” Mayo Clinic Proceedings (1986), 61(9):697-705); and Veronesi, et al. (“Inefficacy of immediate node dissection in stage 1 melanoma of the limbs.” The New England Journal Medicine (1977), 297(12):627-30).

In all of these trials, elective lymphadenectomy did not result in a significant survival benefit. In one of the trials (Balch, et al, 2000), a subgroup analysis indicated that elective lymphadenectomy may benefit patients younger than 60 years of age, especially those with non-ulcerated primary melanomas and melanomas between 1-2 mm in thickness. Based on these results, elective lymphadenectomy for patients with stage I and II melanoma was not advocated and this resulted in a more selective evaluation of the regional lymph nodes and development of the sentinel lymph node biopsy (SLNB) technique.

The sentinel lymph node (SLN) concept is based on the hypothesis that tumor cells from primary melanomas metastasize through the lymphatic system to regional lymph nodes in an orderly fashion and that mapping of the lymphatic system can identify the first or “sentinel” lymph node to receive metastatic tumor cells. This sentinel lymph node will become involved with metastasis before any other node in the regional lymph node basis and if involved will reflect the pathologic status of the entire regional nodal basin. See, Morton et al. (“Technical details of intra-operative lymphatic mapping for early stage melanoma.” Archives Surgery (1992), 127(4):392-9). That study detailed the first evaluation of the SLN concept in patients with stage I melanoma. In this study of 237 lymph node basins in 233 patients, the SLN was identified 82% of the time and it predicted the pathologic status of the nodal basin in 99% of cases. If the sentinel node is free of tumor, then the rest of the nodes in that anatomic region are assumed to be free of tumor and are not removed. Clinically, this is important as there is no survival benefit obtained by removing normal lymph nodes and lymphadenectomy always introduces the possibility of limb paresthesia and edema. SLN biopsy is considered an advance in patient care as it selects only those patients with nodal metastasis who might benefit from lymphadenectomy. Since that preliminary study, substantial progress was made improving and standardizing the techniques for lymphatic mapping and SLNB.

An improvement in the surgeon's ability to identify metastatic disease in lymph nodes will advance surgical therapy by, for example, preserving healthy tissue and minimizing the number of axillary lymph nodes removed. This will improve the patient's quality of life and improve morbidity and long-term mortality. Precise identification of cancer cells that have spread to lymph nodes will enable removal of only the diseased ducts and nodes, while sparing the healthy axillary nodes. The perfunctory removal of all axillary lymph nodes and ducts leads to local edema and increased morbidity. The non-removal of axillary lymph nodes and ducts that contain metastatic cancer cells leads to decreased survival and increased long-term mortality.

Use of a vital blue dye such as 1% isosulfan blue (Lymphazurin®) has been part of the lymphatic mapping and SLNB since its introduction. At the time of operation, 3-5 ml of the vital blue dye is injected intradermally around the intact primary melanoma or the tumor biopsy site. The dye rapidly diffuses into the lymphatic system and is carried by afferent lymphatic trunks to the SLN. An incision is made over the draining nodal basin and the blue afferent lymphatic channels are followed to the first draining lymph node(s), the sentinel lymph nodes. With the use of a vital blue dye, the SLN can be identified in approximately 87% of cases. See Gershenwald, et al. (“Improved sentinel lymph node localization in patients with primary melanoma with the use of radio-labeled colloid.” Surgery (1998), 124(2):203-10). This leaves 13% of patients unable to benefit from a SLN evaluation. Gershenwald et al. demonstrated that SLN identification improved from 87% to 99% when technetium-99m labeled sulfur colloid was combined with the vital blue dye.

To increase the detection rate of SLNs, two additional techniques are commonly used: a) pre-operative lymphoscintigraphy using a technetium-99m labeled sulfur colloid or human albumin radiotracer to better delineate the lymphatic drainage and identify multiple drainage basins; See for example, Essner, et al. (“Standardized probe-directed sentinel node dissection in melanoma.” Surgery (2000), 127(1):26-31) and Pijpers, et al. (“Sentinel node biopsy in melanoma patients: dynamic lymphoscintigraphy followed by intra-operative gamma probe and vital dye guidance.” World Journal Surgery (1997), 21(8):788-92; discussion 93); and b) intraoperative use of a handheld gamma probe to better localize the SLN. Currently, using the vital blue dye technique in combination with a radiotracer identifies the SLN in up to 99% of cases. See for example, Gershenwald, et al. (1998); Morton, et al. (“Validation of the accuracy of intraoperative lymphatic mapping and sentinel lymphadenectomy for early-stage melanoma: a multicenter trial. Multicenter Selective Lymphadenectomy Trial Group.” Annals Surgery (1999), 230(4):453-63; discussion 63-5); and Thompson, et al. (“Location of sentinel lymph nodes in patients with cutaneous melanoma: new insights into lymphatic anatomy.” Journal American College of Surgeons (1999), 189(2):195-204). Based on these findings, most clinicians now recommend using a combined modality approach which is considered the “gold standard” for SLN localization in patients with primary melanoma. Although the technetium-99m labeled sulfur colloid adds a greater detection ability, formal studies have not been reported using this alone. Informal observation finds that one can pick up radioactivity in nodes which are not blue more often than one picks up blue nodes that are not radioactive, but again the ideal situation is to be able to use two tracers at once.

Although 1% isosulfan vital blue dye increases the detection of SLNs when combined with a radiotracer, it has several drawbacks. First, the dye can diffuse throughout the operative wounds making dissection and SLN identification difficult. This is especially concerning if the afferent lymphatic channels are cut. Second, 1% isosulfan blue dye has been associated with an anaphylactoid reaction or a life threatening anaphylactic shock in 0.1-2% of patients undergoing lymphatic mapping and SLNB. See, for example, Cimmino, et al. (“Allergic reactions to isosulfan blue during sentinel node biopsy—a common event.” Surgery (2001), 130(3):439-42); Komenaka, et al. (“Allergic reactions to isosulfan blue in sentinel lymph node mapping.” The Breast Journal (2005), 11(1):70-2); Leong, et al. (“Adverse reactions to isosulfan blue during selective sentinel lymph node dissection in melanoma.” Annals Surgical Oncology (2000), 7(5):361-6); Montgomery, et al. (“Isosulfan blue dye reactions during sentinel lymph node mapping for breast cancer.” Anesthesia Analgesia (2002), 95(2):385-8, table of contents); Raut, et al. (“Incidence of anaphylactoid reactions to isosulfan blue dye during breast carcinoma lymphatic mapping in patients treated with preoperative prophylaxis: results of a surgical prospective clinical practice protocol.” Cancer (2005), 104(4):692-9); and Wilke, et al. (“Surgical complications associated with sentinel lymph node biopsy: results from a prospective international cooperative group trial.” Annals Surgical Oncology (2006), 13(4):491-500). Third, a recent shortage in 1% isosulfan blue has resulted in a decreased access to the compound for patients and clinicians. Lymphazurin has experienced periodic shortages since 2001; was unavailable or severely rationed since August, 2006, and only became commercially available once again as of April 2008.

Fluorescein

Fluorescein is an orange-red powdered compound with the formula C₂₀H₁₂O₅, which exhibits intense greenish-yellow fluorescence in alkaline solution. It has been used extensively in surgery and medicine for decades for diagnostic purposes. Topical fluorescein is routinely used in ophthalmology to assess corneal lesions. See, for example, Kim J (“The use of vital dyes in corneal disease.” Current Opinion Ophthalmology (2000), 11(4):241-7). Intravenous fluorescein is used in vascular surgery to measure vascular perfusion. See, for example, Lund, et al. (“Video fluorescein imaging of the skin: description of an overviewing technique for functional evaluation of regional cutaneous blood perfusion in occlusive arterial disease of the limbs.” Clinical Physiology (Oxford, England) (1997), 17(6):619-33); and in skin and melanoma surgery to assess the viability of skin flaps. See, for example, Casanova, et al. (“Clinical evaluation of flap viability with a dermal surface fluorometer.” Annals Plastic Surgery (1988), 20(2):112-6) and Kreidstein, et al. (“Serial fluorometric assessments of skin perfusion in isolated perfused human skin flaps.” British Journal Plastic Surgery (1995), 48(5):288-93). Intradermal fluorescein injections have been used to identify pedal lymphatics to facilitate lymphangiography. See, for example, Cooper, et al. (“Fluorescein labeling of lymphatic vessels for lymphangiography.” Radiology (1988), 167(2):559-60). This study was designed to look at both the safety and efficacy of using 10% fluorescein mixed 1:1 with 1% lidocaine hydrochloride. Cooper et al. reported on intradermal injection of fluorescein in 1,047 patients without adverse reactions. Dan et al. (“1% lymphazurin vs. 10% fluorescein for sentinel node mapping in colorectal tumors.” Archives Surgery (2004), 139(11):1180-4.) used intramural bowel injection of fluorescein in 120 patients with colon cancer to map the lymphatics in patients with colon cancer. Fluorescein was able to identify the sentinel lymph node in 97% of patients and none of the 120 patients suffered any adverse reactions. A 10% solution of USP sodium fluorescein is currently used in intraoperative procedures to verify venous and arterial anastomosis patency. It is also used to verify the perfusion of microvasculature in plastic and reconstructive surgical procedures. This can be especially important when entire skin flaps must be resected and transplanted.

There is a great need to develop new lymphatic mapping and SLN identification techniques that utilize lower concentrations of mapping agents.

### SUMMARY OF THE INVENTION

The present invention relates generally to the field of fluorescent dyes as a system useful for surgery imaging. More particularly, the present invention relates to systems, methods and kits for exciting fluorescent, phosphorescent or luminescent molecules with light from a light source and detecting the relative fluorescent, phosphorescent, or luminescent light intensity emitted from the fluorescent, phosphorescent, or luminescent molecule. Such systems may be applied as mapping agents for various surgical techniques, such as for cancer surgeries and biopsies.

Thus, in one aspect, the present invention provides a system for visualizing arterial, venous or lymphatic tissue in a mammal, including a human. In accordance with this aspect, the system comprises a dilute solution of a fluorescent, phosphorescent or luminescent dye at a concentration of from about 0.00001% (w/v) to about 1.0% (w/v). The system also comprises a light-emitting component for stimulating the dye to fluoresce, phosphoresce or luminesce. The system further comprises surgical eyeglasses comprising wavelength filters specific (or selective) for filtering out the wavelength of the stimulating light from the light-emitting component. The surgical eyeglasses are transparent at the wavelength range where fluorescence, phosphorescence, and luminescence occurs. In one embodiment, the dilute solution is a stabilized solution of the dye in a biologically-compatible solvent. In another embodiment, the concentration of the dye is from about 0.0001% (w/v) to about 0.1% (w/v). In an additional embodiment, the concentration of the dye is from about 0.001% (w/v) to about 0.01% (w/v). In one embodiment, the system further comprises a light filter to filter the light from the light-emitting component. In another embodiment, the fluorescent dye is fluoroscein.

In one embodiment, the light-emitting component is a light source selected from the group consisting of a laser, a laser diode, a light-emitting diode (LED), an organic light-emitting diode, a fiber-optic light source, a luminous gas discharge and a hot filament lamp. In another embodiment, the light-emitting component is a single LED or an array of LEDs. In an additional embodiment, the LED is a blue LED. In a further embodiment, the blue LED has a peak emission between 430 nm and 490 nm.

In one embodiment, the lenses of the surgical eyeglasses comprise the specific wavelength filters. In another embodiment, the surgical eyeglasses comprise a flipup specific wavelength filter. In an additional embodiment, the surgical eyeglasses have specific wavelength filters mounted on the lenses. In a further embodiment, the specific wavelength filter is a notch filter. In another embodiment, the notch filter is a holographic notch filter. In one embodiment, the notch filter is specific for filtering out light having a wavelength between 430 nm and 490 nm. In another embodiment, the surgical eyeglasses are transparent at a wavelength around 520 nm. In one embodiment, the filter to filter the light from the light-emitting component is a Wratten #47 filter fitted to the light-emitting component.

In a second aspect, the present invention provides a method of detecting the location of a light-emitting material in tissue of a mammal including human. In accordance with this aspect, the method comprises administering a dilute solution of a light emitting material, such as a fluorescent, phosphorescent or luminescent dye, at a concentration of from about 0.00001% (w/v) to about 1.0% (w/v) into the tissue. The method also comprises illuminating the tissue with a light emitted from a light-emitting component to stimulate the dye to fluoresce, phosphoresce or luminesce. The method further comprises detecting the location of the dye within the tissue based on the fluorescence of the dye. In one embodiment, the dilute solution is as described above.

In one embodiment, the light-emitting component further comprises a light filter to filter the light from the light-emitting component. In another embodiment, the light-emitting component is a light source as described above. In one embodiment, the light-emitting component further comprises a probe selectively coupled to it. In another embodiment, the probe may be a hand-held probe, a finger-tip mounted probe, a surgical telescope, an endoscope, a cytoscope, a nephroscope, a bronchoscope, a laryngoscope, a otoscope, an arthroscope, a laparascope, a colonoscopic endoscope or a gastrointestinal endoscope.

In one embodiment, the detection is performed using surgical eyeglasses comprising a wavelength filter specific for filtering out the wavelength of the stimulating light from the light-emitting component. The surgical eyeglasses are transparent at the wavelength range where fluorescence, phosphorescence, and luminescence occurs. In another embodiment, the eyeglasses and specific wavelength filter are as described above. In one embodiment, the light-emitting material is located preferentially in cancerous, neoplastic, dysplastic or hyperplastic tissue. In another embodiment, the light-emitting material is located preferentially in non-cancerous, non-neoplastic, non-dysplastic or non-hyperplastic tissue.

In one embodiment, the method further comprises performing a surgical procedure on the mammal. In another embodiment, the surgical procedure may be lymphatic mapping or sentinel lymph node localization. In an additional embodiment, the method is employed for the surgical treatment of the mammal with neoplasms (cancer), melanoma, basal cell carcinoma and squamous cell carcinoma, breast, esophageal, stomach, pancreatic, colon, small bowel, lung, anal or rectal, uterine, prostate, penile, testicular, head or neck and soft-tissue sarcoma. In one embodiment, the tissue of the mammal is a lumen. In another embodiment, lumen is selected from the group consisting of a fistula tract, vas deferens, cystic duct and common bile duct.

In a third aspect, the present invention provides a method for performing sentinel lymph node (SLN) biopsy in breast cancer and melanoma surgery in a mammal. In accordance with this aspect, the method comprises administering a dilute solution of a fluorescent, phosphorescent or luminescent dye, at a concentration of from about 0.00001% (w/v) to about 1.0% (w/v) into the tissue. The method also comprises illuminating the tissue with a light emitted from a light-emitting component to stimulate the dye to fluoresce, phosphoresce or luminesce. The method further comprises detecting the location of the dye within the tissue based on the fluorescence, phosphorescence or luminescence of the dye. The method also comprises, performing tumor excision to remove the breast cancer or melanoma. In one embodiment, the dilute solution is as described above. In one embodiment, the light-emitting component further comprises a light filter to filter the light from the light-emitting component. In another embodiment, the light-emitting component is a light source as described above. In one embodiment, the light-emitting component further comprises a probe selectively coupled to it. In another embodiment, the probe is as described above. In one embodiment, the detection is performed using surgical eyeglasses comprising a wavelength filter specific for filtering out the wavelength of the stimulating light from the light-emitting component. The surgical eyeglasses are transparent at the wavelength range where fluorescence, phosphorescence, and luminescence occurs. In another embodiment, the eyeglasses and specific wavelength filter are as described above.

In a fourth aspect, the present invention provides a kit for detecting a light-emitting material in a mammal, including a human, during a surgical procedure. In accordance with this aspect, the kit comprises a dilute solution of a fluorescent, phosphorescent or luminescent dye at a concentration of from about 0.00001% (w/v) to about 1.0% (w/v). The kit also comprises a light-emitting component for stimulating the dye to fluoresce, phosphoresce or luminesce. The kit further comprises instructions describing a method of administering the solution in the mammal and using the light-emitting component to visualize the tissue. In one embodiment, the kit may also comprise surgical eyeglasses comprising wavelength filters specific for filtering out the wavelength of the stimulating light from the light-emitting component. The surgical eyeglasses are transparent at the wavelength range where fluorescence, phosphorescence, and luminescence occurs. In another embodiment, the dilute solution is as described above. In one embodiment, the light-emitting component further comprises a light filter to filter the light from the light-emitting component. In another embodiment, the light-emitting component is a light source as described above. In an additional embodiment, the eyeglasses and specific wavelength filter are as described above.

### BRIEF DESCRIPTION OF THE DRAWINGS

FIG. 1 shows an embodiment which incorporates features of the invention, showing a modified Stryker endoscope coupler containing holographic notch filter.

FIG. 2 shows an embodiment which incorporates features of the invention, showing a hexagonal array of 7 Luxeon LED's (3 W each) in illuminator head. The Wratten #47 filter has been removed for this picture.

FIG. 3 shows an embodiment which incorporates features of the invention, showing a complete surgical light stand. In one embodiment, the illumination head is mounted on a sliding head that is connected to the power supply with a flexible coiled-cord. The sliding rail and head can be raised or lowered to match the animal (or patient) under study. All electrical fittings are hospital-grade.

FIG. 4 shows an embodiment which incorporates features of the invention, showing an LED configuration and polymer optics lens. Shown left-to-right: (a) Far left is the latest generation of high-power LED from Philips Luxeon is the “Rebel” series LED, shown as the small yellow object above the unsharpened end of the pencil. The “Rebel” series LED produces as much power as the Luxeon “Star” (second from left), but draws less power and requires less-precise thermal management. (b) Second-to-left is the Luxeon “Star” high-power LED that is available in 3 W and 5 W packages. This model requires precise thermal management and must be mounted on an electrically-isolated heatsink. (c) Third-from-left is an array of 7 Luxeon “Star” 3 W LED's mounted on an electrically-isolated metal-core printed circuit board. Each LED is fitted with a separate optical lens. (d) Far Right is a plastic concentrator lens assembly that will focus the output from 7 separate high-power LED's mounted to a metal-core printed circuit board. This plastic concentrator lens is used to focus the output from 7 LED's into the liquid light guide from the dual-wavelength illuminator for minimally-invasive endoscopy approaches to visualization.

FIG. 5 shows an embodiment which incorporates features of the invention, showing fluorescein in lymphatic vessels is visible through skin of pig. USP Fluorescein (0.01%) can easily be seen through the skin of the pig following subdermal injection, as would be used in a sentinel lymph node biopsy procedure. Using an optimal concentration of fluorescein (in one embodiment, 1,000-fold more dilute than the commercially-available USP preparation sold by Akorn, Inc.) and an optimized blue light LED array for visualization allows fluorescein to be seen before the skin is dissected to access the sentinel lymph node. In this picture, the fluorescein has migrated along the 8-inch path within 4 minutes. The migration of fluorescein can be followed visually and in real-time to direct the surgeon to the sentinel lymph node for excision.

FIG. 6 shows an embodiment which incorporates features of the invention, showing Fluorescein in Lymphatic Vessel after Opening the Skin. Fluorescein in the lymphatic vessel clearly leads the surgeon to the sentinel lymph node that is the first lymph node to drain the nodal tissue basin.

FIG. 7 shows an embodiment which incorporates features of the invention, showing the sentinel lymph node glows brightly when the skin at the end of the fluorescent lymphatic vessel is opened. Fluorescence in the lymphatic trunk has led the surgeon directly to the sentinel lymph node in this example. As applied in breast cancer surgery, this lymph node would be removed and submitted for a detailed examination for cancer by a pathologist. By employing a 1,000-fold dilution of the commercially-available 10% USP sodium fluorescein, coupled with transdermal illumination by 480 nm blue light and visualization through orange glasses, it is possible to see the flow of fluorescein in the lymphatic vessels without darkening the operating room. The frames shown in FIG. 8 depict the time course of fluorescein as it migrates through the lymphatic vessels of a 40 kg dark-skinned swine. The fluorescein migrates rapidly through the lymphatic vessels. After only 10 seconds, the fluorescein that has migrated through the lymphatic vessels appears to stop, as visualized through the skin. The lymphatic vessel dives away from the skin at this point, and ends at the sentinel lymph node in a cluster of nodes. The process of localizing the sentinel lymph node by this technique is fast (10-30 seconds after subdermal injection), and would not require a pre-operative injection of visualization agent.

FIG. 8 shows an embodiment which incorporates features of the invention, showing time-course of fluorescein migration in a lymphatic vessel. Rapid migration of 0.01% fluorescein can be observed in the lymphatic vessel of a 40 kg dark-skinned swine. The fluorescein can be observed through the skin without creating a surgical wound. The arrow in the 10-second frame indicates the location of the sentinel lymph node, as proved by dissecting the tissue immediately below this point.

FIG. 9 shows a pair of surgical eyeglasses having a notch filter as the lenses. The notch filter is specific for light having a wavelength between 430 nm and 490 nm

FIG. 10 shows groin incision where lymphatic mapping with 0.01% fluorescein has identified the location of the sentinel lymph node.

FIG. 11 shows 0.01% flourescein is stimulated and seen fluorescing the the lymphatic trunks. Lymphazurin™ (darker central portion of the lymphatic trunks) co-localizes with fluorescein in the lymphatic trunks. The fluorescent lymphatic trunk is seen through the intact skin to the right of the incision.

### DETAILED DESCRIPTION OF THE INVENTION

Definitions

As used herein, the following definitions shall apply unless otherwise indicated.

“Mammal” refers to any animal classified as a mammal, including humans, domestic, and farm animals, and pet animals, such as cats, dogs, horses, pigs, sheeps, cows, etc. A particular preferred mammal is a human.

“Patient” or “Subject” refers to human and non-human animals, especially mammals.

“Pharmaceutically acceptable salt” refers to pharmaceutically acceptable salts of a compound, such as a dye, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, the term “fluorescence” refers to the emission of a photon from an excited electronic singlet state of a molecule or atom.

As used herein, the term “phosphorescence” refers to the emission of a photon from an excited electronic triplet state of a molecule or atom.

As used herein, the term “luminescence” refers to the emission of a photon from an excited electronic state of a molecule or atom.

The present invention relates generally to the field of fluorescent, phosphorescent or luminescent dyes as a system useful for surgery imaging. More particularly, the present invention relates to systems, methods and kits for exciting fluorescent, phosphorescent or luminescent molecules with light from a light source and detecting the relative fluorescent, phosphorescent, or luminescent light intensity emitted from the fluorescent, phosphorescent, or luminescent molecule. Such systems may be applied as mapping agents for various surgical techniques, such as for cancer surgeries and biopsies.

Suitable fluorescent compounds (dyes) that can be used in the present invention include, but are not limited to, cobalafluors, fluorescein, fluorescein-5-EX succinimidyl ester (FSE), methoxycoumarin, naphthofluorescein, BODIPY 493/503, BODIPY FL, BODIPY R6G, BODIPY 530/550, BODIPY TMR, BODIPY 564/570, BODIPY 576/589, BODIPY 581/591, BODIPY TR, Cascade Blue, Dansyl, dialkylaminocoumarin, 4′,5′-dichloro-2′,7′-dimethyloxyfluorescein, 2′,7′-dichlorofluorescein, eosin, eosin F3S, erythrosin, hydroxycoumarin, lissamine rhodamine B, NBD, Oregon Green 488, Oregon Green 500, Oregon Green 514, PyMPO, pyrene, rhodamine 6G, rhodamine green, rhodamine red, rhodamine 123, rhodol green, 2′,4′,5′,7′-tetrabromosulfonefluorescein, tetramethylrhodamine (TMR), Texas Red, X-rhodamine, Cy2 dye, Cy3 dye, Cy5 dye, Cy5.5 dye, Cy7 dye, IC Green, riboflavin, a chelating moiety that binds a lanthanide ion or a quantum dot structure. Suitable phosphorescent compounds (dyes) that can be used in the present invention include, but are not limited to, eosin Y, platinum octaethylporphyrin (PtOEP), platinum octaethylporphyrin ketone (PtOEPK), platinum tetrakis (pentafluorophenyl) porphyrin (PtTFPP), palladium octaethylporphyrin (PdOEP), palladium octaethylporphyrin ketone (PdOEPK), palladium tetrakis (pentafluorophenyl) porphyrin (PdTFPP) and Ru (dpp) 3C12 (dpp+4,7-diphenyl-1, 10-phenanthroline).

In the description which follows, the terms “fluorescent compound” or “fluorescent dye” or “fluorescence” are used to describe the invention. However, it is understood that phosphorescent compound or dye or luminescent compound or dye can be used in place of fluorescent compound or dye and phosphorescence or luminescence can be used in place of fluorescence. In addition, the use of fluorescein as the light-emitting material (i.e., fluorescent dye) is for illustration purposes and is not intended to be limiting. Specific details concerning particular light-emitting components and detectors (e.g., surgical eyeglasses with specific wavelength filters) are provided with respect to fluorescein. It is understood that the skilled artisan will select light-emitting components and detectors on the basis of the fluorescent, phosphorescent or luminescent dye chosen for use in the system, methods and kits of the present invention.

The present invention relates generally to the field of fluorescent dyes, as a system, a method and a kit useful for surgery imaging. Such systems, methods and kits may be applied as mapping agents for various surgical techniques, such as for cancer surgeries and biopsies. In a preferred embodiment, the present invention relates to highly dilute solutions of fluorescein, or similarly photoactive molecule, as a superior alternative to currently utilized dye markers for tumor excision and sentinel lymph node biopsy in breast cancer and melanoma surgery. Accordingly, the present invention provides provided systems, methods and kits to map and visualize photoactive compounds in vasculature and tissue of human and animal patients. The present invention further provides systems, methods and kits for detecting light-emitting material in a human or animal patient during a surgical procedure. The systems, methods and kits may be used for lymphatic mapping and sentinel lymph node biopsies as described herein.

Thus, in one aspect, the present invention provides a system for visualizing arterial, venous or lymphatic tissue in a mammal, including a human. In accordance with this aspect, the system comprises a dilute solution of a fluorescent, phosphorescent or luminescent dye at a concentration of from about 0.00001% (w/v) to about 1.0% (w/v). The system also comprises a light-emitting component for stimulating the dye to fluoresce, phosphoresce or luminesce. The system further comprises surgical eyeglasses comprising wavelength filters specific for filtering out the wavelength of the stimulating light from the light-emitting component. The surgical eyeglasses are transparent at the wavelength range where fluorescence, phosphorescence, and luminescence occurs. In one embodiment, the dilute solution is a stabilized solution of the dye in a biologically-compatible solvent. In another embodiment, the concentration of the dye is from about 0.0001% (w/v) to about 0.1% (w/v). In an additional embodiment, the concentration of the dye is from about 0.001% (w/v) to about 0.01% (w/v). In one embodiment, the system further comprises a light filter to filter the light from the light-emitting component. In another embodiment, the fluorescent dye is fluorescein.

In one embodiment, the light-emitting component is a light source selected from the group consisting of a laser, a laser diode, a light-emitting diode (LED), an organic light-emitting diode, a fiber-optic light source, a luminous gas discharge and a hot filament lamp. In another embodiment, the light-emitting component is a single LED or an array of LEDs. In an additional embodiment, the LED is a blue LED. In a further embodiment, the blue LED has a peak emission between 430 nm and 490 nm.

In one embodiment, the lenses of the surgical eyeglasses comprise the specific wavelength filters. In another embodiment, the surgical eyeglasses comprise a flipup specific wavelength filter. In an additional embodiment, the surgical eyeglasses have specific wavelength filters mounted on the lenses. In a further embodiment, the specific wavelength filter is a notch filter. In another embodiment, the notch filter is a holographic notch filter. In one embodiment, the notch filter is specific for filtering out light having a wavelength between 430 nm and 490 nm. In another embodiment, the surgical eyeglasses are transparent at a wavelength around 520 nm. In another embodiment, the filter to filter the light from the light-emitting component is a Wratten #47 filter fitted to the light-emitting component.

In a second aspect, the present invention provides a method of detecting the location of a light-emitting material in tissue of a mammal including human. In accordance with this aspect, the method comprises administering a dilute solution of a light emitting material, such as a fluorescent, phosphorescent or luminescent dye, at a concentration of from about 0.00001% (w/v) to about 1.0% (w/v) into the tissue. The method also comprises illuminating the tissue with a light emitted from a light-emitting component to stimulate the dye to fluoresce, phosphoresce or luminesce. The method further comprises detecting the location of the dye within the tissue based on the fluorescence of the dye. In one embodiment, the dilute solution is as described above.

In one embodiment, the light-emitting component further comprises a light filter to filter the light from the light-emitting component. In another embodiment, the light-emitting component is a light source as described above. In one embodiment, the light-emitting component further comprises a probe selectively coupled to it. In another embodiment, the probe may be a hand-held probe, a finger-tip mounted probe, a surgical telescope, an endoscope, a cytoscope, a nephroscope, a bronchoscope, a laryngoscope, a otoscope, an arthroscope, a laparascope, a colonoscopic endoscope or a gastrointestinal endoscope.

In one embodiment, the detection is performed using surgical eyeglasses comprising a wavelength filter specific for filtering out the wavelength of the stimulating light from the light-emitting component. The surgical eyeglasses are transparent at the wavelength range where fluorescence, phosphorescence, and luminescence occurs. In another embodiment, the eyeglasses and specific wavelength filter are as described above. In one embodiment, the light-emitting material is located preferentially in cancerous, neoplastic, dysplastic or hyperplastic tissue. In another embodiment, the light-emitting material is located preferentially in non-cancerous, non-neoplastic, non-dysplastic or non-hyperplastic tissue.

In one embodiment, the method further comprises performing a surgical procedure on the mammal. In another embodiment, the surgical procedure may be lymphatic mapping or sentinel lymph node localization. In an additional embodiment, the method is employed for the surgical treatment of the mammal with neoplasms (cancer), melanoma, basal cell carcinoma and squamous cell carcinoma, breast, esophageal, stomach, pancreatic, colon, small bowel, lung, anal or rectal, uterine, prostate, penile, testicular, head or neck and soft-tissue sarcoma. In one embodiment, the tissue of the mammal is a lumen. In another embodiment, lumen is selected from the group consisting of a fistula tract, vas deferens, cystic duct and common bile duct.

In one embodiment, certain fluorescent compounds and their compositions may be employed in the methods provided herein. The light may be provided by an arc lamp, a hot filament emitter, a laser, a light-emitting diode, or a fiber-optic light source with appropriate filter. Specific fluorescent compounds and the light sources that may be employed according to the present methods as described herein have been described in U.S. Pat. No. 6,905,884 and U.S. Patent Application Publication Nos. 2004/0082863 and 2007/0269837 among others, the disclosure of each is hereby incorporated herein by reference in their entirety.

In a third aspect, the present invention provides a method for performing sentinel lymph node (SLN) biopsy in breast cancer and melanoma surgery in a mammal. In accordance with this aspect, the method comprises administering a dilute solution of a fluorescent, phosphorescent or luminescent dye, at a concentration of from about 0.00001% (w/v) to about 1.0% (w/v) into the tissue. The method also comprises illuminating the tissue with a light emitted from a light-emitting component to stimulate the dye to fluoresce, phosphoresce or luminesce. The method further comprises detecting the location of the dye within the tissue based on the fluorescence, phosphorescence or luminescence of the dye. The method also comprises, performing tumor excision to remove the breast cancer or melanoma. In one embodiment, the dilute solution is as described above. In one embodiment, the light-emitting component further comprises a light filter to filter the light from the light-emitting component. In another embodiment, the light-emitting component is a light source as described above. In one embodiment, the light-emitting component further comprises a probe selectively coupled to it. In another embodiment, the probe is as described above. In one embodiment, the detection is performed using surgical eyeglasses comprising a wavelength filter specific for filtering out the wavelength of the stimulating light from the light-emitting component. The surgical eyeglasses are transparent at the wavelength range where fluorescence, phosphorescence, and luminescence occurs. In another embodiment, the eyeglasses and specific wavelength filter are as described above.

In a fourth aspect, the present invention provides a kit for detecting a light-emitting material in a mammal, including a human, during a surgical procedure. In accordance with this aspect, the kit comprises a dilute solution of a fluorescent, phosphorescent or luminescent dye at a concentration of from about 0.00001% (w/v) to about 1.0% (w/v). The kit also comprises a light-emitting component for stimulating the dye to fluoresce, phosphoresce or luminesce. The kit further comprises instructions describing a method of administering the solution in the mammal and using the light-emitting component to visualize the tissue. In one embodiment, the kit may also comprise surgical eyeglasses comprising wavelength filters specific for filtering out the wavelength of the stimulating light from the light-emitting component. The surgical eyeglasses are transparent at the wavelength range where fluorescence, phosphorescence, and luminescence occurs. In another embodiment, the dilute solution is as described above. In one embodiment, the light-emitting component further comprises a light filter to filter the light from the light-emitting component. In another embodiment, the light-emitting component is a light source as described above. In an additional embodiment, the eyeglasses and specific wavelength filter are as described above.

In addition to the embodiments described above, another embodiment of the present invention provides a system for visualizing arterial, venous or lymphatic tissue in a mammal, comprising: a stabilized solution of a fluorescent, phosphorescent or luminescent dye dissolved in a biologically-compatible solvent at a concentration of 0.01% (w/v) or less; a light-emitting component for stimulating the dye to fluoresce, phosphoresce or luminesce; and instructions describing a method of administering the dye in the mammal and using the light-emitting component to visualize arterial, venous or lymphatic tissue in the mammal. In an additional embodiment, there is provided a system for detecting light-emitting material in a tissue of a mammal during a sentinel lymph node biopsy procedure, comprising: an array of blue light-emitting diodes (LEDs) with a peak emission between 430 nm and 490 nm; a Wratten #47 filter fitted to the array; a solution of fluorescein dissolved in isotonic (0.9% w/v) saline, having a concentration of 0.01% (w/v); and instructions describing a method of administering the solution in the mammal and using the LEDs to visualize the tissue.

In addition to the embodiments described above, another embodiment of the present invention provides a kit for detecting light-emitting material in a mammal during a surgical procedure, comprising: an array of blue light-emitting diodes (LEDs) with a peak emission between 430 nm and 490 nm; a Wratten #47 filter fitted to the array; eyeglasses with specific wavelength filters; a solution of fluorescein dissolved in isotonic (0.9% w/v) saline at a concentration of 0.01% (w/v); and instructions describing a method of administering the fluorescein solution in the mammal and using the LEDs to visualize arterial, venous or lymphatic tissue in the mammal.

In addition to the embodiments described above, another embodiment of the present invention provides a method of detecting the location of a light-emitting material in tissue of a mammal, the method comprising: administering a stabilized solution of a fluorescent dye dissolved in a biologically-compatible solvent at a concentration of 0.01% or less (w/v), into the tissue; illuminating the tissue with a light emitted from a light-emitting component; and detecting the location of the dye within the tissue based on the fluorescence of the dye.

In another embodiment, the visualization method of the present application may be used to visualize the condition of various tissues or lumens in a mammal. In one aspect, the method disclosed herein may be used in a fistulography procedure, wherein the dye is injected, such as to the external opening of the fistula, followed by visualization of the fistula tract to determine condition, obstruction or blockages of the fistula tract. Such procedure may be used to identify the primary opening of the fistula or with multiple fistulae, may be used to identify secondary tracts or missed primary tract openings. In another aspect, the procedure may also be used to determine the success of a tubal ligation or a vasectomy procedure. In another aspect, the procedure may be used in conjunction or in place of a laparoscopic procedure for the direct or indirect visualization of the peritoneal cavity, ovaries, outside of the tubes and uterus. In another aspect, the visualization procedure may also be used to assist in determining the condition, presence or absence of obstruction of the cystic duct or the common bile duct. In addition, the procedure may be used to determine the success or completion of a cholecystectomy procedure, wherein the cystic duct is clipped two or three times and a cut is made between the clips, freeing the gallbladder to be taken out.

In a particular aspect, the method provides the detection of fluorescent (cancer-targeted or non-targeted) in a lymph node. In another aspect, there is provided a method of detecting the location of fluorescent material in a sample using the above-described apparatuses. The sample may be biological tissue and the fluorescent material may be located preferentially in cancerous, neoplastic, dysplastic or hyperplastic tissue. The fluorescent material may be located in surrounding or structurally integrated non-cancerous, non-neoplastic, non-dysplastic, or non-hyperplastic tissue. In yet another aspect, there is provided a method of removing fluorescent material in a sample using the above-described apparatuses. The sample may be biological tissue and the fluorescent material may be located preferentially in cancerous, neoplastic, dysplastic or hyperplastic tissue. The fluorescent material may be located preferentially in non-cancerous, non-neoplastic, non-dysplastic or non-hyperplastic tissue. In another aspect, there is provided a method of removing non-fluorescent material in a sample using the above-described apparatuses. The sample may be biological tissue and the fluorescent material may be located preferentially in cancerous, neoplastic, dysplastic or hyperplastic tissue. The fluorescent material may be located preferentially in surrounding or structurally integrated non-cancerous, non-neoplastic, non-dysplastic or non-hyperplastic tissue. In yet another aspect, there is provided a method of removing cancerous, neoplastic, dysplastic or hyperplastic tissue from a subject, the method comprising: providing to the subject a fluorescent dye that preferentially localizes to cancerous, neoplastic, dysplastic or hyperplastic tissue, detecting the level of relative fluorescent intensity in the subject, and laser ablating the tissue in which the relative fluorescent intensity indicates the preferential localization of the fluorescent dye. In one aspect, he removal or destruction of a sample or a portion of a sample include, but are not limited to, electrocautery devices or scalpels, and ultrasonic cutters or ablators.

Fluorescein as Fluorescent Dye

As disclosed herein, a fluorescein composition may be used in place of the isosulfan blue dye. When fluorescein is injected under the skin, it is rapidly taken up by local lymph nodes which then fluoresce when activated with a light source. Using eyewear specific to the fluorescent signal, the nodes are easily seen and then removed. The safety profile of fluorescein shows it to be a very well tolerated material as documented over the number of years when employed in numerous medical procedures. Typical safety precautions are written for a 10% (w/v) fluorescein solution delivered intravenously. A differentiator of the composition of the present application is that highly diluted solutions of fluorescein of between 0.00001% (w/v) and 1% (w/v) are utilized that are only 1:1,000,000 to 1:10 of the concentration of commercially available solution. In a preferred embodiment, fluorescein of between 0.0001% (w/v) and 0.1% (w/v) are utilized. In a more preferred embodiment, fluorescein of between 0.001% (w/v) and 0.01% (w/v) are utilized. As such, any adverse reactions listed should be significantly fewer and lesser in degree that those reported for intravenous use of a 10% solution of fluorescein.

One aspect of present application provides that the fluorescein composition may be prepared from a solution of fluorescein in water, saline or a biologically-compatible solvent. Fluorescein (as the free-acid form) can be titrated to a physiologically-compatible pH by use of any biologically-compatible base (most commonly Na⁺, K⁺). A particular aspect of the present application provides the concentration of aqueous fluorescein that is to be injected to be less than 5% (w/v) to produce optimal images. In another aspect, the concentration of fluorescein is less than 1% (w/v) and greater than 1×10⁻⁵% (w/v). In another aspect, the diluted fluorescein can be injected in any manner, including (but not exclusively) subdermally, intradermally, intramuscularly, intravenously and intrathecally.

In another aspect, fluorescein may be stimulated with any light source that emits light from 230 nm to 1,500 nm. The high limit on wavelength is to include the use of multi-photon excitation. In one aspect, the light source for excitation of fluorescein emits light between 400 nm and 510 nm. The light source can produce coherent and non-coherent illumination. The preferred light source is a light-emitting diode, with a peak emission between 430 nm and 490 nm. As an example, diluted fluorescein as disclosed herein may be used for any medical procedure that requires lymphatic mapping and/or sentinel lymph node localization, including human or animal patients with neoplasms (cancer), melanoma, basal cell carcinoma and squamous cell carcinoma. As further examples of the invention, the localization of lymphatic vessels and lymph nodes may benefit patients, either human or animal, with cancers including (but not exclusively) breast, skin (melanoma), bone, connective tissue, digestive organs (esophageal, stomach, small intestine, large intestine, rectum, colon, liver), pancreatic, colon, small bowel, lung, anal or rectal, uterine, prostate, gynecological (ovarian, prostate, uterine, cervical, vulval), urinary organs (bladder, kidney), penile, testicular, head or neck (lip, tongue, mouth, pharynx), eye, brain and central nervous system, endocrine glands (thyroid), lymph tissue, and soft-tissue sarcoma.

As further examples of the present methods as described herein, diluted fluorescein compositions of the present application may be used in other medical procedures including, but not limited to 1) assessment of microvascular perfusion in the reattachment of body parts, for ischemic bowel, and for myocutaneous flaps in reconstructive surgery; 2) testing the integrity of surgical anastomoses at all sites including, but not limited to, the esophagus, bile duct, and all lower anterior anastomoses; 3) testing for occult perforation of the gastrointestinal tract; 4) testing for integrity of vascular anastomoses; 5) identification of the common bile duct during laparoscopic cholecystectomy; 6) identification of the ureter during pelvic operations; 7) assessment of patency of a re-anastomosis of the vas deferens; 8) assessment of patency of the fallopian tubes; 9) identification of nerves that might be damaged in an operation; 10) visualization of the cerebrospinal fluid during back surgery to detect a defect in the dural sac around the spinal cord; 11) guiding the practitioner to the location of a leak in the dura from a spinal tap for a therapeutic blood patch; and 12) addition to the fluid used to inflate breast implants and other reconstructive devices to detect leaking implants.

Option for Minimally-Invasive Visualization of Fluorescein without Endoscopic Components

An example of an embodiment of the present application comprises a method of detecting the presence and location of a light-emitting material in a sample. In one aspect, light of a proper wavelength to stimulate the light-emitting material to produce fluorescent, phosphorescent, or luminescent light is directed onto a particular portion of the sample. Fluorescent, phosphorescent or luminescent light emitted from that portion of the sample, if any, is collected and the intensity of the fluorescent, phosphorescent or luminescent light is used to provide audio and visual cues to the practitioner of the method. In this manner, the practitioner can distinguish between parts of sample that contain or do not contain a light-emitting material.

In a further example of the method, the sample may comprise biological tissue. In a further example, the fluorophore may be preferentially located in cancerous, neoplastic, dysplastic or hyperplastic tissue. Thus, the practice of one example of a method according to the present application enables a practitioner to distinguish between normal tissue and cancerous, neoplastic, dysplastic or hyperplastic tissue. Applicants have found conditions under which fluorescein in lymphatic vessels can also be visualized transdermally and the location of the sentinel lymph node can be found in the tissue immediately below the point where the lymphatic vessel dives away from the skin and the fluorescein disappears from the surface. This technique decreases the invasiveness of the SLN biopsy approach by precisely identifying the location of the sentinel lymph node.

Illumination and Visualization Components

It will be appreciated by one of ordinary skill in the art that any type of light-emitting component may be used so long as it provides a frequency of light capable of stimulating a fluorescent, phosphorescent or luminescent emission from the light-emitting material. Examples of light-emitting components suitable for use in the present invention, include, but are not limited to, lasers, laser diodes, light-emitting diodes, organic light-emitting diodes, fiber-optic light sources, luminous gas discharges, hot filament lamps and similar light sources. In one particular aspect, the method provides the use of a light-emitting diode or laser diode. In one embodiment, the light-emitting component can be a flashlight, other hand-held independently powered light source or a pencil-shaped device powered by a power box containing the light source. Such light-emitting components may contain a single LED or an array of LEDs. Because it is known that LEDs and other light sources degrade during sterilization, the flashlight or other light-emitting components can be disposable.

A switchable dual-light source can be used in the systems and methods of the present invention. In one embodiment, a dual-light source has been developed with a hexagonal matrix of 7 LED's that is coupled into a polymethylmethacrylate polymer optic focusing lens. For example, a white light source also may be provided allowing for more familiar full color viewing. Such full color viewing is useful for anatomical orientation within the host and for viewing on the video monitor. A dual light source that includes both the light-emitting component and the white light source may be utilized to provide an easy mechanism for rapid switching between non-white light for fluorescence viewing and white light for conventional viewing. Such switching may be accomplished by any mechanism such as, for example, voice-actuated switching, a mechanically-operated switch (e.g., a foot pedal), an optically-operated switch, or an electronically-operated switch. For example, a commercially-available fiber optic dual-lamp xenon light source may be modified by replacing one of the lamps with a blue diode laser. Another variant could be a device that includes two internal light sources (one white and one non-white) and a mirror or prism under mechanical or electromechanical control to switch between the two light sources.

In a further embodiment of the present application, localization detectors of more than one system can be combined, including detectors that sense radiation in the visible light wavelength, or other localizer techniques such as gamma radiation or sound waves. Exemplifying embodiments comprise combination of a fluorescent detector with a radiation detector, or combination of a fluorescent detector with an ultrasound transducer. Alternatively, the detector can be one which locks onto a modulated light signal, such as by detecting light having a changing amplitude with a known frequency. Similar embodiments would be realized by those skilled in the art.

As will be additionally appreciated by one of ordinary skill in the art, any type of light filter may be used such that the filter has the capability to remove, block, absorb, reflect or deflect a portion of the light passing there-through. Examples of filters suitable for use in the present application, include, but are not limited to, notch filters, holographic notch filters, long-pass filters, short-pass filters, interference filters, absorptive neutral density filters, reflective neutral density filters, infrared filters, prisms, gratings and mirrors.

An example of an embodiment according to the present application comprises a method of detecting the presence and location of a light-emitting material in a sample. Light of a proper wavelength to stimulate the light-emitting material to produce fluorescent, phosphorescent or luminescent light is directed onto a particular portion of the sample. Fluorescent, phosphorescent, or luminescent light emitted from that portion of the sample, if any, is collected and the intensity of the fluorescent, phosphorescent, or luminescent light is used to provide audio and visual cues to the practitioner of the method. In this manner, the practitioner of the method can distinguish between parts of sample that contain or do not contain a light-emitting material. In a further example of a method according to the present invention, the sample may comprise biological tissue. In yet a further example, the fluorophore may be preferentially located in cancerous, neoplastic, dysplastic or hyperplastic tissue. Thus, the practice of one example of a method according to the present invention enables a practitioner to distinguish between normal tissue and cancerous, neoplastic, dysplastic or hyperplastic tissue.

A further example of an embodiment comprises a method of removing light-emitting material or non- light-emitting material from a sample. Light of a proper wavelength to stimulate the light-emitting material to produce fluorescent, phosphorescent or luminescent light is directed onto a particular portion of the sample. Fluorescent, phosphorescent or luminescent light emitted from that portion of the sample, if any, is collected and the intensity of the fluorescent, phosphorescent, or luminescent light, if beyond a predetermined user defined or otherwise established threshold, is used to activate a laser or other device that ablates or otherwise destroys the portion of the sample which is fluorescing. In this manner, the practitioner of the method can distinguish between parts of sample that contain or do not contain a light-emitting material and ablate or otherwise destroy portions of the sample, depending on the wishes of the operator, which do or do not emit a threshold level of fluorescent, phosphorescent, or luminescent light. In a further example of a method according to the present invention, the sample may comprise biological tissue. In yet a further example, the fluorophore may be preferentially located in cancerous, neoplastic, dysplastic or hyperplastic tissue. Thus, in one embodiment, the method enables a practitioner to distinguish between normal tissue and cancerous, neoplastic, dysplastic or hyperplastic tissue and to selectively remove or destroy parts or substantially all of the cancerous, neoplastic, dysplastic or hyperplastic tissue as desired.

In a further embodiment, the fluorescence system described herein may be combined as part of a larger system wherein the fluorescent output triggers the firing of an ablating source of light energy such as a laser. For human malignancies that are treated with light energy (photodynamic therapy), a photophore that concentrates in the tissue to be destroyed by light would fluoresce when stimulated, and initiate the destroying energy source (ultrasound, gamma knife or light). For ablation of tattoos in the skin, similar mechanisms may be applied by placing a photophore into the cells containing the tattoo pigment to be destroyed, and incorporating a device that can generate increasing wavelengths appropriate to the desired fluorescent output, and then apply laser energy when the pigments in the tattoos fluoresce, luminesce or alternatively emit any form of energy in the light spectrum.

Kits

Also provided are kits for administration of the compounds, or pharmaceutical formulations comprising the compound that may include a dosage amount of the compound, as disclosed herein. Kits may further comprise suitable packaging and/or instructions for use of the compound. Kits may also comprise a means for the delivery of the fluorescent compound or other components and devices as described herein.

Additionally, the compounds of the present invention can be assembled in the form of kits. The kit provides the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch or inhalant. The syringe may contain a 30 gauge needle which may contain a bend at, for example, the hub. It has been found that there is less spreading of the fluorescein composition using a 30 gauge needle. It has been found that the bend in the needle assists in delivering the fluorescent composition intra-dermally and parallel to the skin, particularly for use in the methods relating to melanoma. The bend in the needle also aids in avoiding contamination of the area. The kits may also comprise surgical eyeglasses having a wavelength filter specific for the fluorescent dye being used. The surgical eyeglasses could be prescription or non-prescription. The wavelength filter could be a holographic notch filter as the lenses of the eyeglasses. FIG. 9 shows a pair of surgical eyeglasses having a notch filter as the lenses. The kits may comprise appropriate instructions for the preparation and administration of the composition and side effects of the compositions, and other relevant information. The instructions may be in any suitable format, including, but not limited to, printed matter, videotape, computer readable disk or optical disc, and combinations thereof.

In one embodiment, there is provided a kit comprising a compound selected from the compounds of the invention, packaging, and instructions for use. In another embodiment, there is provided a kit comprising the pharmaceutical formulation comprising a compound or composition selected from the compounds of the invention and at least one pharmaceutically acceptable excipient, diluent, preservative, stabilizer, or mixture thereof, packaging and instructions for use.

Pharmaceutical Compositions

Pharmaceutical compositions comprising the compounds described herein (or salts thereof) can be manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries which facilitate processing of the compounds into preparations which can be used as described. Such preparations may also be prepared to provide enhanced protection against factors including photochemical degradation or air-oxidation.

The fluorescent compound can be formulated in the pharmaceutical compositions per se, or in the form of a hydrate, solvate or pharmaceutically acceptable salt, as described herein. Typically, such salts are more soluble in aqueous solutions than the corresponding free acids and bases, but salts having lower solubility than the corresponding free acids and bases may also be formed.

In one embodiment, there is provided a pharmaceutical formulation comprising a compound selected from the compounds of the invention, as described herein, and at least one pharmaceutically acceptable excipient, diluent, preservative, stabilizer or mixture thereof. Thus, in a specific embodiment, the fluorescent compound (and the various forms described herein, including pharmaceutical formulations comprising the compounds (in the various forms)) can be used in the methods as described herein in animal subjects, including humans. The methods generally comprise administering to the subject an amount of a compound of the invention, or a salt, or hydrate thereof, effective for the method described herein. In one embodiment, the subject is a non-human mammal, including, but not limited to, bovine, horse, feline, canine, rodent or primate. In another embodiment, the subject is a human.

The compounds and composition of the present application can be administered in accordance with customary cancer diagnostic, detection, prediction, prognostication, monitoring or characterization methods known in the art. For example, the compound and composition can be administered intravenously, intrathecally, intratumorally, intramuscularly, intralymphatically, or orally. Typically, an amount of the compound or composition of the present application may be admixed with a pharmaceutically acceptable carrier. The carrier may be used in a variety of forms depending on the form of preparation desired for administration, e.g., oral, parenteral (e.g., intramuscular, intraperitoneal, intravenous, ICV, intracisternal injection or infusion, subcutaneous injection, or implant), and epidural. The compositions may further contain antioxidizing agents, stabilizing agents, preservatives and the like. Examples of techniques and protocols may be found in Remington: The Science and Practice of Pharmacy, 21st Ed., Ed. D.B. Troy, Lippincott, Williams & Wilkins, Baltimore, 2006, the disclosure of which is incorporated herein in its entirety.

Useful injectable preparations include sterile suspensions, solutions or emulsions of the compound(s) and compositions in aqueous. The compositions may also contain formulating agents, such as suspending, stabilizing, and/or dispersing agents. The formulations for injection can be presented in unit dosage form, e.g., in ampoules or in multidose containers, and may contain added preservatives. The injectable formulation can be provided in powder form for reconstitution with a suitable vehicle, including but not limited to sterile pyrogen free water, buffer, and dextrose solution, before use. To this end, the composition may be dried by any art-known technique, such as lyophilization, and then reconstituted prior to use.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution, and isotonic sodium chloride solution.

For ocular administration, the fluorescent compound(s) can be formulated as a solution, emulsion, suspension, etc., suitable for administration to the eye. A variety of vehicles suitable for administering compounds to the eye are known in the art. Specific non-limiting examples are described in U.S. Pat. No. 6,261,547; U.S. Pat. No. 6,197,934; U.S. Pat. No. 6,056,950; U.S. Pat. No. 5,800,807; U.S. Pat. No. 5,776,445; U.S. Pat. No. 5,698,219; U.S. Pat. No. 5,521,222; U.S. Pat. No. 5,403,841; U.S. Pat. No. 5,077,033; U.S. Pat. No. 4,882,150; and U.S. Pat. No. 4,738,851.

Advantages of Fluorometric Lymphatic Mapping

The present invention demonstrates that the intraoperative use of fluorescein (fluorescent lymphatic mapping) improves the SLN biopsy procedure and improve the therapeutic outcome for the following reasons: 1) a smaller incision is needed to define the lymphatic anatomy and identify the SLN; 2) the SLN can be identified through layers of overlying tissue with greater precision, thereby reducing operative time (the current SLN identification procedure demands a complete dissection to see the blue dye and depends upon imprecise measurements of radioactivity); 3) the use of ionizing radiation can be eliminated; 4) the 1% chance of a life-threatening anaphylactic event caused by the injection of Lymphazurin® can be avoided; 5) fluorescein is already widely distributed and familiar to surgeons, having been used for lymphatic mapping in colon cancer patients; and 6) fluorescein is relatively inexpensive when compared to Lymphazurin® (Lymphazurin® alone costs $108 per treatment versus $2.10 for fluorescein).

The application of lymphatic mapping, SLN identification, and eventually tumor margin identification in accordance with the present invention provides more general applications for the sensitive detection of USP fluorescein with sub-millimeter accuracy.

It has previously been shown that Cy5-cobalamine bioconjugate injected intradermally into the hind limb of pigs is able to identify inguinal sentinel lymph nodes. See, McGreevy, et al. (“Minimally invasive lymphatic mapping using fluorescently labeled vitamin B12.” The Journal of surgical research (2003), 111(1):38-44). As shown herein, fluorescein injected intradermally into the limb of pigs is also able to identify the sentinel lymph node. Additionally, when 1% isosulfan blue is injected in the same location as fluorescein, the two detection techniques co-localizes in the afferent lymphatics and the sentinel lymph node. The fluorescent signal from fluorescein provides improved detection of the afferent lymphatic and the sentinel lymph node compared to 1% isosulfan blue. Moreover, fluorescein fluorescence is clearly visualized transdermally and enables an improved localization of the sentinel lymph node prior to performing a skin incision. This transdermal fluorescence may enable elimination of the radiotracer in sentinel lymph node detection.

Significant Advantages of the Dilute Fluorescein Formulation

As shown herein, the data obtained in pigs shows that 0.01% sodium fluorescein (a 1,000-fold dilution of commercially-available 10% USP sodium fluorescein that is packaged for parenteral administration) fluoresces brighter than undiluted 10% USP sodium fluorescein. 10% fluorescein has a dark-orange color and has a sufficiently high optical density such that nearly all of the photons that impinge on the solution are absorbed in the first few microns of the solution lightpath. Another photo-physical property that is inherent to highly-fluorescent and highly-colored dyes is self-quenching, in which a dye molecule in the excited electronic state undergoes resonance energy transfer with another dye molecule, with the eventual dissipation of excitation energy through internal conversion. These properties exist for nearly all fluorescent dyes with a small-to-moderate Stokes shift between the excitation and emission maximum.

The present invention provides many potential benefits to mammalian, including human, subjects. For example, a subject with skin cancer can benefit from knowing the location of lymphatic vessels and lymph nodes, including patients with melanoma, basal cell carcinoma, and squamous cell carcinoma. A subject with breast cancer can benefit from knowing the location of lymphatic vessels and lymph nodes. A subject with esophageal cancer can benefit from knowing the location of lymphatic vessels and lymph nodes. A subject with stomach cancer can benefit from knowing the location of lymphatic vessels and lymph nodes. A subject with pancreatic cancer can benefit from knowing the location of lymphatic vessels and lymph nodes. A subject with colon cancer can benefit from knowing the location of lymphatic vessels and lymph nodes. A subject with small bowel cancer can benefit from knowing the location of lymphatic vessels and lymph nodes. A subject with lung cancer can benefit from knowing the location of lymphatic vessels and lymph nodes. A subject with anal or rectal cancer can benefit from knowing the location of lymphatic vessels and lymph nodes. A subject with uterine cancer can benefit from knowing the location of lymphatic vessels and lymph nodes. A subject with prostate cancer can benefit from knowing the location of lymphatic vessels and lymph nodes. A subject with penile cancer can benefit from knowing the location of lymphatic vessels and lymph nodes. A subject with testicular cancer can benefit from knowing the location of lymphatic vessels and lymph nodes. A subject with head and neck cancer can benefit from knowing the location of lymphatic vessels and lymph nodes. A subject with soft-tissue sarcomas cancer can benefit from knowing the location of lymphatic vessels and lymph nodes. In addition, a subject with any medical use for which the practitioner needs to know the location of the lymphatic anatomy can benefit from the use of diluted fluorescein to identify the location of lymphatic vessels and lymph nodes. The injection and visualization can be performed in the operating room, in the clinic, in a pre-operative room, or in any other suitable setting.

The preferred fluorescent dye, i.e., fluorescein, provides additional advantages. For example, fluorescein can be used to assess microvascular perfusion in the reattachment of body parts, for ischemic bowel, and for myocutaneous flaps in reconstructive surgery. Fluorescein can be used to test for the integrity of surgical anastomoses at all sites, including, but not limited to the esophagus, the bile duct, and all lower anterior anastomoses. Fluorescein can be used to test for occult perforation of the gastrointestinal tract. Fluorescein can be used to test for the integrity of vascular anastomoses. Fluorescein can be used to identify the common bile duct during laparoscopic cholecystectomy. Fluorescein can be used to identify the ureter during pelvic operations. Fluorescein can be used to assess patency of a re-anastomosis of the vas deferens. Fluorescein can be used to identify nerves that might be damaged in an operation by binding the fluorescent agent to the nerves. Fluorescein can be used to visualize the cerebrospinal fluid during back surgery to detect a defect in the dural sac around the spinal cord. Fluorescein can be used to guide the practitioner to the location of a leak in the dura from a spinal tap for a therapeutic blood patch. Fluorescein can be added to the fluid used to inflate breast implants and other reconstructive devices to detect leaking breast implants by placing concentrated fluorescein into the implant; a leak would cause the fluorescing compound to leak into the blood and then into the urine where fluorescence would indicate a leaking implant.

### Examples

The present invention is described by reference to the following Examples, which is offered by way of illustration and is not intended to limit the invention in any manner. Standard techniques well known in the art or the techniques specifically described below were utilized.

### Example 1

### Initial Studies

Illumination devices are based upon high intensity blue light-emitting diodes (LEDs) and emit an intense band of light that is centered at 480 nm and has a half-height bandwidth of about 90 nm. The half-height bandwidth is narrowed to about 70 nm with truncation of a higher-wavelength tail by placing a Wratten #47 filter in front of the focusing and collimating lens of the illuminator housing. The blue light emitted by this configuration is ideally suited for the transdermal excitation of fluorescein and can be effectively blocked by specially-selected yellow-orange lenses that can be mounted in photographic filter holders for photographic documentation purposes or in eyeglass frames for wear by the surgeon or the user.

A holographic notch filter (Kaiser Optical) to remove the intense, but spectrally narrow band of blue light from the LED light sources, was fitted into a surgical telescope camera adapter. The camera adapter design used by Stryker Endoscopy was chosen for modification, as it was much easier to modify and build from than camera adapters manufactured by other companies. The compact surgical telescope fitted with the holographic notch filter is shown in FIG. 1.

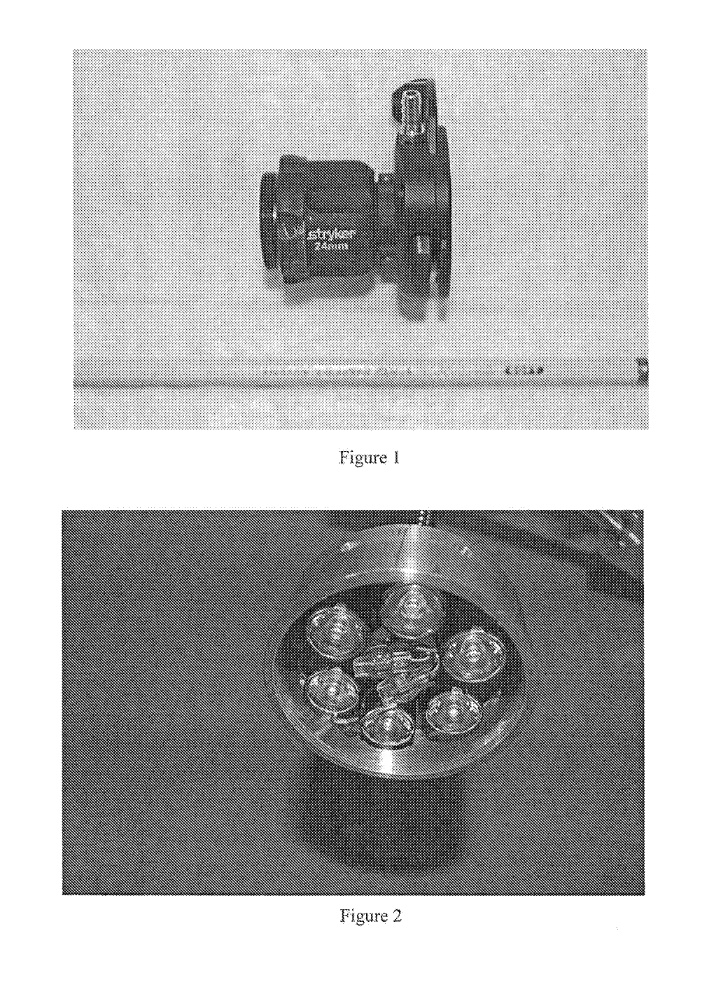

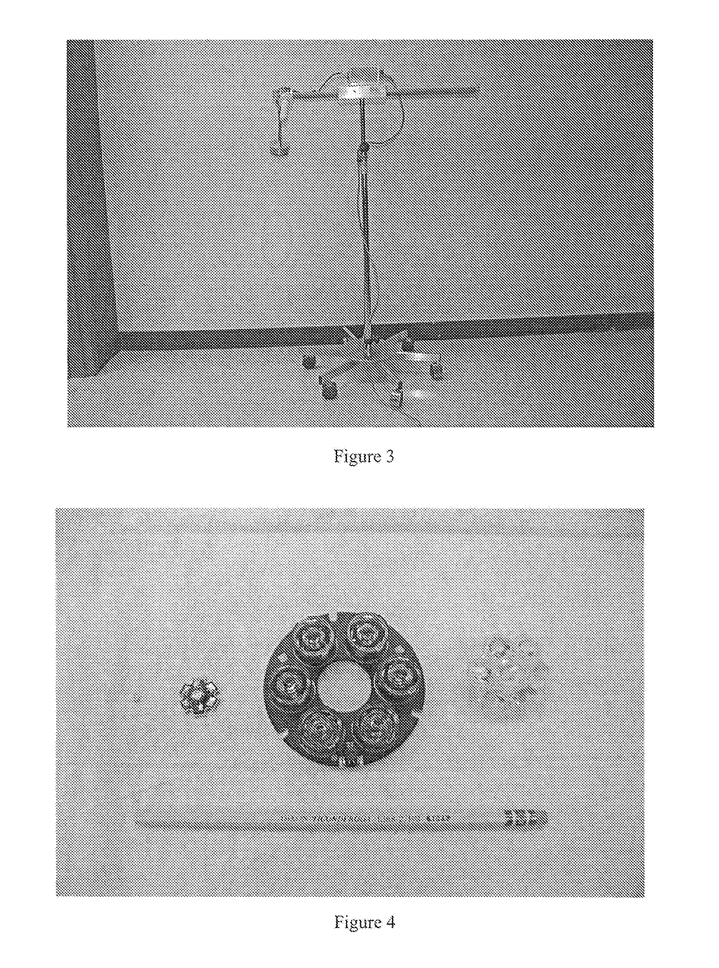

Light sources with high-intensity 3- and 5-watt Luxeon LumiLEDs were manufactured and evaluated. An array of 7 Luxeon 3-watt LEDs mounted to a PCB in a hexagonal packing array (FIG. 2) were found to be the best light source for the excitation of fluorescein in open surgical dissection. The LEDs produce intense blue light that is optimal for fluorescein excitation when further filtered with a Wratten #47 filter affixed to the front of the array. This light source is mounted onto a sliding support that accepts a standard sterile plastic handle (Karl Storz, Inc.) for manipulation by the surgeon (FIG. 3). When used with orange spectacles (U60, NoIR Medical), the devices for open dissection are sufficient to proceed with the project.

A switchable dual-light source has been developed with a hexagonal matrix of 7 LED's that is coupled into a polymethylmethacrylate polymer optic focusing lens shown in FIG. 4 (Polymer Optics, Ltd, UK), and further focused into a liquid light guide. The transmission through a liquid light guide is greater than 70% over 2 m.

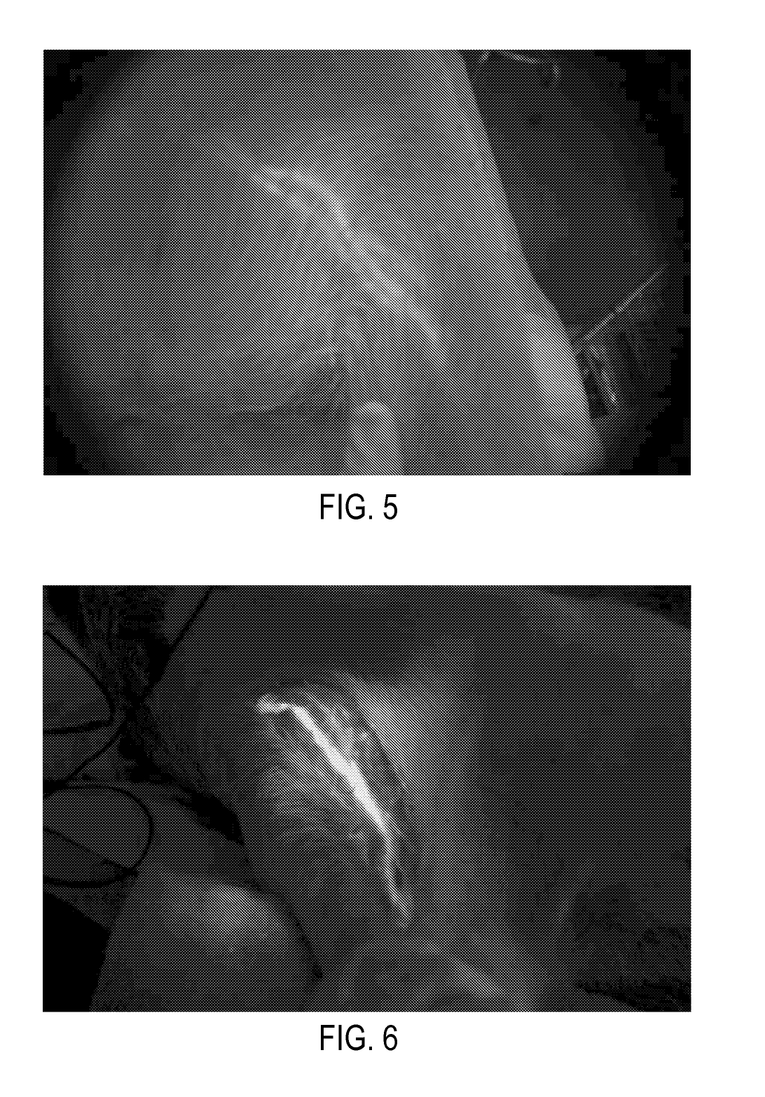

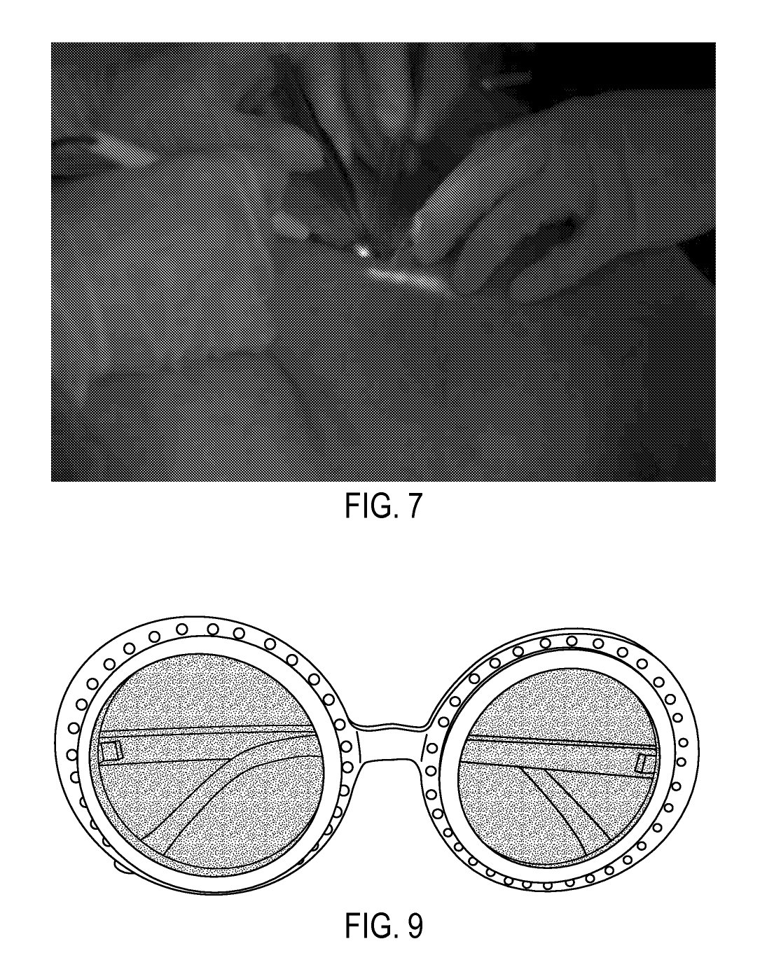

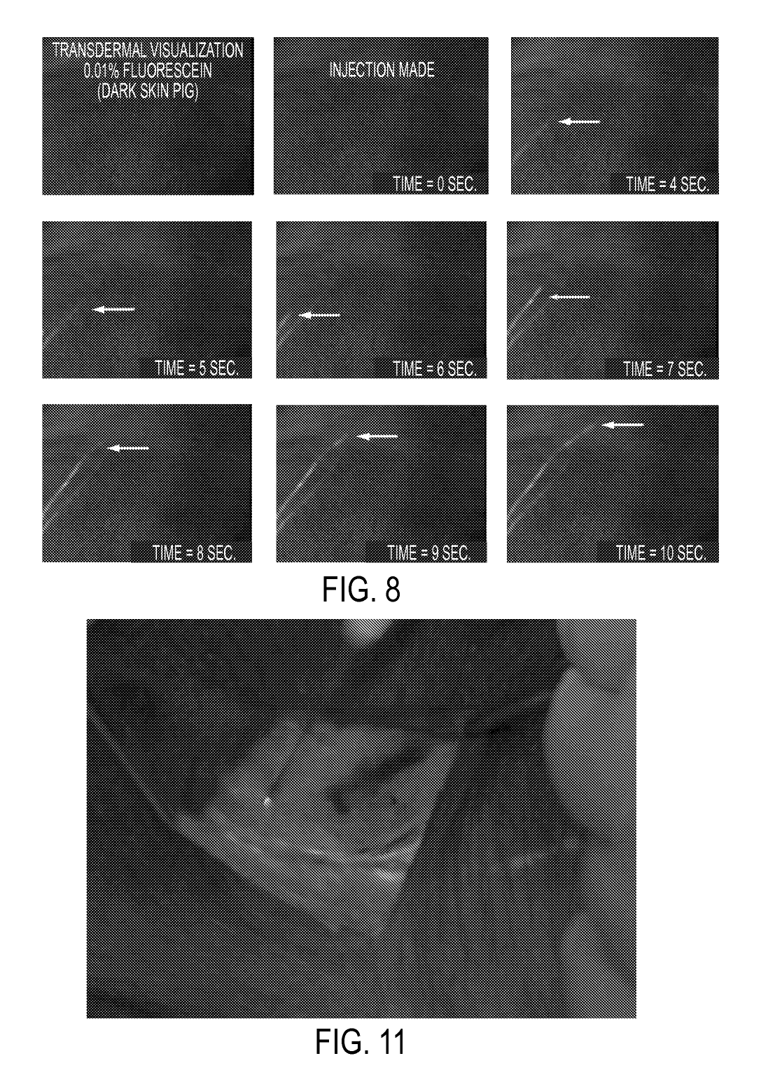

The device for illuminating fluorescein for lymphatic mapping in an open dissection was initially evaluated in 3 pigs. Detection of fluorescein in lymphatic vessels and in sentinel lymph nodes was spectacularly successful, with images shown in FIGS. 5, 6 and 7. Selection of the optimal wavelength to excite fluorescein makes visualization of the lymphatic vessels possible through the skin. FIG. 8 shows the time-course of fluorescein migration in a lymphatic vessel. This is noteworthy, as it may permit the use of the fluorescein composition for sentinel lymph node detection in an open dissection, but with visualization in the lymphatic trunks prior to making a surgical incision. Furthermore, according to the present methods described herein, the optimal concentration of fluorescein is about 0.01% for injection, rather than the commercially available 10% fluorescein. This is a significant improvement over other published attempts to use fluorescein for lymphatic mapping, where 1% or 10% fluorescein was employed.

### Example 2

### Materials and Methods

Fluorescein: 10% Fluorescein USP (Mallinckrodt Baker, Inc., Phillipsburg, N.J.) was diluted in normal saline to concentrations of 0.001%, 0.01%, and 0.1%. The diluted fluorescein was injected (0.5-1.0 ml) into the dermis of the distal forelimb and hindlimb of swine using a 1.0 ml tuberculin syringe. The dose and number of dermal injections varied according to the result. Sometimes the first injection illuminated a large lymphatic trunk easily visible through the skin. This happened more often in the hindlimb than the forelimb. Occasionally, several injections were needed to illuminate a channel. And sometimes, no lymphatic channel filled from the dermal injection. This happened in forelimbs only. Most often, after less than three injections, a lymphatic channel drained from the limb to nodes as seen through the skin and led to the nodal basin containing the sentinel node. In this model, the sentinel nodes were not visible through the skin. An open surgical wound exposed the sentinel nodes which fluoresced brightly every time that a major trunk was identified. Lymphazurin™ (1% isosulfan blue, Covidien, Norwalk, Conn.) at a volume of 0.4 to 1.0 ml was injected intradermally to evaluate co-localization with the fluorescein identified lymphatic channels.

Fluorescent stimulation and emission detection: Illumination devices based upon high-intensity blue light-emitting diodes (LED's) that emit an intense band of light that is centered at 480 nm and has a half-height bandwidth of about 90 nm were used. The half-height bandwidth was narrowed to about 70 nm with truncation of a higher-wavelength tail by placing a Wratten #47 filter in front of the focusing and collimating lens of the illuminator housing. The blue light emitted by this configuration is ideally suited for the transdermal excitation of fluorescein and can be blocked by B+W #023 3X Multi-Reflection Coating (MRC) yellow-orange lenses that can be mounted in photographic filter holders for photographic documentation purposes or in eyeglass frames for wear by the surgeon.

The porcine model of lymphatic mapping: Post-adolescent female pigs, with an average weight of 30 kg were housed at the University of Utah Animal Resource Center. After a minimum 5 day acclimatization period, pigs were fasted for 12 hours. Anesthetic induction was perfomed with an IM injection of TKX Solution (4.4 mg/kg Telazol, 2.2 mg/kg Ketamine and 2.2 mg/kg Xylazine). The pigs were then intubated, placed on mechanical ventilation and anesthetized with 1-2% Isoflurane. They were monitored by an AnimalResource Center Technician for signs of light anesthesia, such as movement, breathing or rapid heart rate. After a surgical plane of anesthesia was established, an IV was placed in a marginal ear vein and secured. A normal saline drip of 22 ml/kg/min was established to maintain hydration of the pigs and to provide a route for drug administration. Animals were euthanized with Beuthanasia while under a deep surgical plane of general aneshesia. This method is consistent with the recommendations of the Panel on Euthansia of the Americn Veterinary Medical Association and the Association for Assessment and Accreditation of Laboratory Animal Care International (AAALAC).

### Example 3

### Lymphatic Mapping

A total of five swine were studied utilizing all four limbs; the total number of lymphatic nodal basins evaluated was 20. Of the ten fluorescein hindlimb injections, nine (90%) showed lymphatic trunks under the skin leading to the SLN. In the forelimbs available for study, seven fluorescein injections were performed and only one (14%) developed visible fluorescence leading to a SLN. Because of the poor visualization of lymphatic trunks with fluorescein in the forelimbs of swine, we stopped injecting forelimbs in the last animals studied. In the forelimbs, lymphazurin was injected four times and failed to lead to a SLN. We feel that the forelimb lymphatic drainage in the swine may lead to the deep lymphatics more often than the superficial ones as is seen reliably in the swine hindlimb (Wallace, et al. “Lymphoseek: a molecular imaging agent for melanoma sentinel lymph node mapping.” Annals of Surgical Oncology (2007) 14(2):913-21.).

Injection method: We found that a completely dermal injection under steady pressure produced the best result. Lymphatic trunks were often visible immediately with rapid progression of the fluorecent marker under the skin in the lymphatic. If this did not occur, moving the injection site a centimeter away from the original site was usually successful. Injections into the subcutaneous tissue did not reliably drain into lymphatics.

Fluorescent lymphatic mapping: We found that the intensity of the fluorescent imaging allowed the operating surgeon to follow the lymphatic under the skin to the groin. An incision was made where the signal disappeared and in that location the glowing lymphatic trunk could be followed to the SLN. FIG. 10 is a lymphatic trunk leading to a groin skin incision, and the fluorescent signal within the SLN incision. A strong fluorescent signal was seen in a bisected SLN (data not shown).

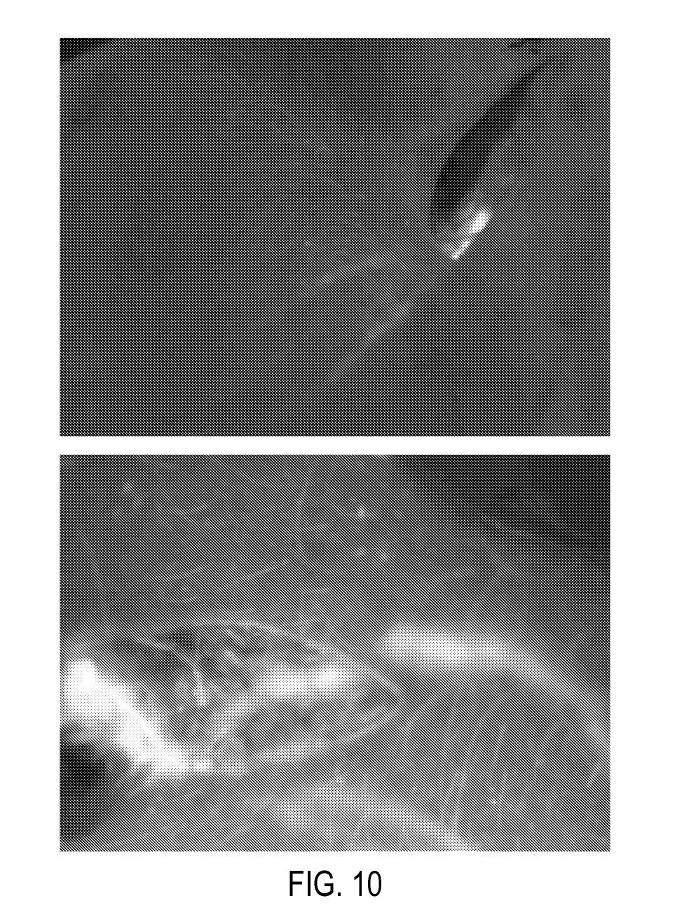

Validation with simultaneous lymphazurin injection: In the first two swine, we injected 1.0 ml of Lymphazurin™ to confirm that the lymphatics illuminated by the fluorescein were the same as the ones filled by the blue dye commonly used by surgeons in SLN operations. In each case, the two markers co-localized in the same lymphatic trunks and drained to the same SLN. We also found that Lymphazurin™ quenched the fluorescence of the fluorescein where they were simultaneously present. FIG. 11 shows lymphatic trunks containing both mapping agents.

These results demonstrate that highly dilute fluorescein is superior to the non-fluorescent blue dye, Lymphazurin® that is traditionally used for lymphatic mapping and lymph node localization. A key to the success of using fluorescein as an intra-operative lymphatic mapping agent is the use of the illumination and visualization system described herein that allows for the direct visualization of the fluorescein lymphatic mapping agent through the skin.

### Example 4

### Human Melanoma Clinical Trial

Patients with cutaneous malignant melanoma with a Breslow thickness of 0.75 mm or greater, or Clark level IV/V involvement, or ulceration were enrolled in the trial. All patients underwent preoperative lymphoscintigraphy with 99m-Technetium labeled sulfur colloid up to 24 hours before the operation. The normally used intraoperative vital blue dye was replaced by fluorescein. The initial starting dose of fluorescein was 0.001% and 1 ml was injected intradermally intraoperatively in 4 locations around the melanoma biopsy site. At this does, sentinel lymph node fluorescence was identified in 3 out of 5 patients (60%). As per protocol, since fluorescence was observed in less than 80% or patients, the dose was increased to 0.01%. At this dose, fluorescence was observed in 4 out of 5 patients, and as per protocol, this dose was determined to be the appropriate dose. The 0.01% dose was used in the subsequent phase II study. Sentinel lymph node fluorescence has been observed in all 51 patients studied in the phase II study to date.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the systems, methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description and by reference to the drawings and figures. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Thus, the described embodiments are illustrative and should not be construed as restrictive.

The present invention has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

1-48. (canceled)

49. A system for visualizing arterial, venous or lymphatic tissue in a mammal, comprising:
a dilute solution of a fluorescent, phosphorescent or luminescent dye at a concentration of (a) from about 0.00001% (w/v) to about 1.0% (w/v) or (b) from about 0.0001% (w/v) to about 0.1% (w/v) or (c) from about 0.001% (w/v) to about 0.01% (w/v), preferably wherein the dye is a fluorescent dye which is preferably fluorescein;
a light-emitting component for stimulating the dye to fluoresce, phosphoresce or luminesce;
optionally a light filter to filter the light from the light-emitting component;
surgical eyeglasses comprising a wavelength filter selective for filtering out the wavelength of the stimulating light from the light-emitting component, wherein the eyeglasses are transparent to the fluorescence, phosphorescence or luminescence of the dye; and
optionally instructions describing a method of administering the solution in the mammal and using the light-emitting component and the surgical eyeglasses to visualize the tissue.

50. The system of claim 49, wherein the light-emitting component is a light source selected from the group consisting of a laser, a laser diode, a light-emitting diode (LED), an organic light-emitting diode, a fiber-optic light source, a luminous gas discharge and a hot filament lamp, preferably wherein the light-emitting component is a single LED or an array of LEDs, optionally wherein the LED is a blue LED, optionally wherein the blue LED has a peak emission between 430 nm and 490 nm.

51. The system of claim 49, wherein the selective wavelength filter is a notch filter, optionally a holographic notch filter, optionally specific for filtering out light having a wavelength between 430 nm and 490 nm.

52. The system of claim 51, wherein the lenses of the surgical eyeglasses comprise the specific wavelength filter or the surgical eyeglasses comprise a flipup specific wavelength filter.

53. A system for detecting light-emitting material in a tissue of a mammal during a sentinel lymph node biopsy procedure, comprising:
a single blue light-emitting diode (LED) with a peak emission between 430 nm and 490 nm or an array of blue LEDs with a peak emission between 430 nm and 490 nm;
optionally a Wratten #47 filter fitted to the LED or array of LEDs;
a solution of fluorescein dissolved in isotonic (0.9% w/v) saline, having a concentration from about 0.001% (w/v) to about of 0.01% (w/v) or preferably about 0.01% (w/v);
surgical eyeglasses comprising a wavelength filter selective for filtering out the wavelength of the stimulating light from the light-emitting component having a wavelength between 430 nm and 490 nm, wherein the eyeglasses are transparent to the fluorescence of the fluorescein; and
optionally instructions describing a method of administering the fluorescein solution in the mammal and using the LED or array of LEDs and surgical eyeglasses to visualize the tissue.

54. A kit for visualizing arterial, venous or lymphatic tissue in a mammal, comprising:
a dilute solution of a fluorescent, phosphorescent or luminescent dye at a concentration of (a) from about 0.00001% (w/v) to about 1.0% (w/v) or (b) from about 0.0001% (w/v) to about 0.1% (w/v) or (c) from about 0.001% (w/v) to about 0.01% (w/v), preferably wherein the dye is a fluorescent dye which is preferably fluorescein;
a light-emitting component for stimulating the dye to fluoresce, phosphoresce or luminesce;
optionally a light filter to filter the light from the light-emitting component;
surgical eyeglasses comprising a wavelength filter selective for filtering out the wavelength of the stimulating light from the light-emitting component, wherein the eyeglasses are transparent to the fluorescence, phosphorescence or luminescence of the dye; and.
instructions describing a method of administering the solution in the mammal and using the light-emitting component and the surgical eyeglasses to visualize the tissue.

55. The kit of claim 54, wherein the light-emitting component is a light source selected from the group consisting of a laser, a laser diode, a light-emitting diode (LED), an organic light-emitting diode, a fiber-optic light source, a luminous gas discharge and a hot filament lamp, preferably wherein the light-emitting component is a single LED or an array of LEDs, optionally wherein the LED is a blue LED, optionally wherein the blue LED has a peak emission between 430 nm and 490 nm.

56. The kit of claim 54, wherein the selective wavelength filter is a notch filter, optionally a holographic notch filter, optionally specific for filtering out light having a wavelength between 430 nm and 490 nm, preferably wherein the lenses of the surgical eyeglasses comprise the specific wavelength filter or the surgical eyeglasses comprise a flipup specific wavelength filter.

57. A kit for detecting light-emitting material in a tissue of a mammal during a sentinel lymph node biopsy procedure or during a surgical procedure, comprising:
a single blue light-emitting diode (LED) with a peak emission between 430 nm and 490 nm or an array of blue LEDs with a peak emission between 430 nm and 490 nm;
optionally a Wratten #47 filter fitted to the LED or array of LEDs;
a solution of fluorescein dissolved in isotonic (0.9% w/v) saline, having a concentration from about 0.001% (w/v) to about of 0.01% (w/v) or preferably about 0.01% (w/v);
surgical eyeglasses comprising a wavelength filter selective for filtering out the wavelength of the stimulating light from the light-emitting component having a wavelength between 430 nm and 490 nm, wherein the eyeglasses are transparent to the fluorescence of fluorescein; and
instructions describing a method of administering the fluorescein solution in the mammal and using the LED or array of LEDs and surgical eyeglasses to visualize the tissue.

58. A method of detecting the location of a light-emitting material in tissue of a mammal, the method comprising:
administering a solution of a fluorescent, phosphorescent or luminescent dye dissolved in a biologically-compatible solvent at a concentration (a) from about 0.00001% (w/v) to about 1.0% (w/v) or (b) from about 0.0001% (w/v) to about 0.1% (w/v) or (c) from about 0.001% (w/v) to about 0.01% (w/v) into the tissue, preferably wherein the dye is a fluorescent dye which is preferably fluorescein, preferably wherein the tissue of a mammal is a lumen, preferably wherein the lumen is selected from the group consisting of a fistula tract, vas deferens, cystic duct and common bile duct;
illuminating the tissue with a light emitted from a light-emitting component to stimulate the dye to fluoresce, phosphoresce or luminesce; and
detecting the location of the dye within the tissue based on the fluorescence, phosphorescence or luminescence of the dye.

59. The method of claim 54, wherein the detection is performed using surgical eyeglasses comprising a wavelength filter selective for filtering out the wavelength of the stimulating light from the light-emitting component, wherein the eyeglasses are transparent to the fluorescence, phosphorescence or luminescence of the dye, preferably wherein the selective wavelength filter is a notch filter, optionally a holographic notch filter, optionally specific for filtering out light having a wavelength between 430 nm and 491 nm, preferably wherein the lenses of the surgical eyeglasses comprise the specific wavelength filter or the surgical eyeglasses comprise a flipup specific wavelength filter.

60. The method of claim 58, wherein the light-emitting component is a light source selected from the group consisting of a laser, a laser diode, a light-emitting diode (LED), an organic light-emitting diode, a fiber-optic light source, a luminous gas discharge and a hot filament lamp, preferably wherein the light-emitting component is a single LED or an array of LEDs, optionally wherein the LED is a blue LED, optionally wherein the blue LED has a peak emission between 430 nm and 490 nm.

61. The method of claim 58, further comprising a probe configured to be selectively coupled to the light-emitting component, where the probe is selected form the group consisting of hand-held probes, finger-tip mounted probes, surgical telescopes, endoscopes, cytoscopes, nephroscopes, bronchoscopes, laryngoscopes, otoscopes, arthroscopes, laparascopes, colonoscopic endoscopes and gastrointestinal endoscopes.

62. The method of claim 58, further comprising performing a surgical procedure on the mammal, preferably wherein the surgical procedure is selected from the group consisting of lymphatic mapping or sentinel lymph node localization; and the method is employed for the surgical treatment of the mammal with neoplasms (cancer), melanoma, basal cell carcinoma and squamous cell carcinoma, breast, esophageal, stomach, pancreatic, colon, small bowel, lung, anal or rectal, uterine, prostate, penile, testicular, head or neck and soft-tissue sarcoma.

63. A method for performing sentinel lymph node (SLN) biopsy tissue in breast cancer and melanoma surgery in a mammal, the method comprising:
administering a solution of a fluorescent, phosphorescent or luminescent dye dissolved in a biologically-compatible solvent at a concentration (a) from about 0.00001% (w/v) to about 1.0% (w/v) or (b) from about 0.0001% (w/v) to about 0.1% (w/v) or (c) from about 0.001% (w/v) to about 0.01% (w/v) into the tissue, preferably wherein the dye is a fluorescent dye which is preferably fluorescein;
illuminating the tissue with a light emitted from a light-emitting component to stimulate the dye to fluoresce;
detecting the location of the dye within the tissue based on the fluorescence of the dye; and
performing tumor excision to remove the cancer or melanoma.

64. The method of claim 63, wherein the detection is performed using surgical eyeglasses comprising a wavelength filter selective for filtering out the wavelength of the stimulating light from the light-emitting component, wherein the eyeglasses are transparent to the fluorescence of the dye, preferably wherein the selective wavelength filter is a notch filter, optionally a holographic notch filter, optionally specific for filtering out light having a wavelength between 430 nm and 490 nm, preferably wherein the lenses of the surgical eyeglasses comprise the specific wavelength filter or the surgical eyeglasses comprise a flipup specific wavelength filter.

65. The method of claim 63, wherein the light-emitting component is a light source selected from the group consisting of a laser, a laser diode, a light-emitting diode (LED), an organic light-emitting diode, a fiber-optic light source, a luminous gas discharge and a hot filament lamp, preferably wherein the light-emitting component is a single LED or an array of LEDs, optionally wherein the LED is a blue LED, optionally wherein the blue LED has a peak emission between 430 nm and 490 nm.
